(19) United States
(12) Patent Application Publication
(10) Pub. No.: US 2016/0051712 A1
(43) Pub. Date: Feb. 25, 2016

(54) Method and Device for Generating a Sterile Area for an Operation, Examination or Treatment of an Object, in Particular of a Person

(71) Applicant: Adelheid MIRWALD, (US)

(72) Inventor: Guido RIESS, Munich (DE)

(21) Appl. No.: 14/780,713

(22) PCT Filed: Mar. 26, 2014
(86) PCT No.: PCT/DE2014/100102

§ 371 Date: Sep. 28, 2015

(30) Foreign Application Priority Data
Mar. 28, 2013 (DE) 10 2013 103 248.6

(51) Int. Cl. A61L 2/14

(52) U.S. Cl. CPC A61L 2/14

(57) ABSTRACT

The invention relates to a method for generating a sterile area for an operation, examination or treatment of at least a partial area of an object, in particular of a person, in which method cold plasma gas is emitted into the area by at least one plasma generator. The invention further relates to a device for carrying out such a method.

The invention relates to a method and an apparatus for generating a sterile area for an operation, examination, or treatment of at least one partial area of an object, in particular a person. Such apparatuses and methods are used as specified in the invention in environments like hospitals in which it is necessary to establish conditions that, for hygienic or even medical reasons, are as sterile as possible (and therefore freed of living microorganisms, including their dormant stages—spores for example). In addition, applications in veterinary medicine and in the cleaning or sterilization of articles are conceivable.

For example, DE 20 2009 000 537 U1 and DE 20 2008 018 264 U1 propose treating workpiece surfaces or cavities of substrates by means of a so-called cold atmospheric plasma, before the workpieces are coated, in order to remove contamination from the surface and in order to condition and activate the surfaces.

Recently, the use of plasma, particularly cold plasma, has been discussed increasingly as a possible application for (human) medicine. For example, in Bild der Wissenschaft, no. 1/2009, p. 52 the control of foot fungus and the treatment of burn wounds, which inherently become easily infected, are cited as possible applications.

In this envisaged treatment and in the treatment of workpiece surfaces, the use of a plasma jet at a location that, when seen in static terms, constitutes a single point is proposed.

The problematical aspects of plasma sterilization are the temperature that occurs as well as the ozone that results when the plasma is generated (for example as a consequence of UV radiation), which is not only unpleasant for an individual or a patient but can also have adverse health consequences (the gas is an irritant).

The object of the invention is therefore to provide a method for generating a sterile area (i.e. one that at least extends across a surface) and an apparatus therefor that make it possible to perform an operation, examination, or treatment on at least a partial area of an object, in particular a person, in a safe and simple manner without subjecting the person to health risks or without damaging an object.

This object is achieved according to the invention with a method having the elements of claim 1 and an apparatus having the elements of claim 4.

According to the invention, a sterile area, in particular a sterile field for an operation, examination, or any desired treatment—referred to below as an operating field—is generated through the use of an apparatus that comprises at least one plasma gas generator. As specified in the invention, a sterile area, in particular a sterile field, represents an area that extends beyond a wetting at a single point, which is statically covered at a given point in time during a treatment period or which possesses sterilized plasma in a sufficient concentration.

A known use of a so-called plasma torch, on the other hand, essentially represents a wetting at a single point when considered in static terms, even when the plasma torch traverses a line or a small area, and in this wetting at a single point other areas that have not yet been traversed, or areas that have already been traversed are not yet or no longer covered by plasma. Therefore, with this use it is not possible to treat an area or a field that extends beyond an area that essentially is limited to a single point. Furthermore, the length of such a plasma jet would be too short to permit an enlargement of the treatment area (instead of a tip or a point) by increasing the distance to the workpiece.

In order to generate a sterile area, the apparatus of the invention has, for example, an at least partially surrounding housing at the edge of the area, in which preferably a plurality of plasma generators are arranged in such a way that they emit plasma gas in the direction of the interior of the area, as needed in the form of pulses or continuously, so that an adequate concentration of sterilizing plasma gas is present at all times during a treatment. The required concentration can be achieved in the area by means of an appropriately predefined discharge (quantity) of plasma (depending on the shape and size of the area to be sterilized—referred to below as the sterile area).

Of course it is also conceivable that an adequate or desired concentration of plasma could be detected with a suitable sensor and that the sterile area preferably could be released for a treatment once the desired predefined concentration is reached, and/or an alarm signal could be output if the concentration were exceeded. It is also conceivable that the discharge of plasma could be controlled hereby, (for example in the form of a closed-loop threshold control system or closed-loop threshold range control system) in order to ensure a desired concentration above a threshold or within a range during a treatment. Here, a plasma generation shut-off can also be provided if an ozone limit value is exceeded, in particular as a result of turbulence in the plasma field (caused, for example, by excessive operating surgeon movements in the sterile area) in the immediate vicinity of the sterile area, for example greater than 0.1 ppm.

Here, the housing may enclose the field at least partially (open or circumferentially), for example in a U shape, as a polygon, or in the form of a track, which is curved as desired or straight. Of course it would also be conceivable to configure a partial area of the housing to be pivotable or insertable, so that the housing could also be opened and closed. It is further conceivable to configure the housing in the form of a grid, so that webs holding plasma generators could also lie within the area.

In one embodiment of the invention, the housing is closed in an annular shape (for example in the form of a hollow ring). This annular shape may, in addition to being a circular ring, may be in any desired annular shape, for example: polygon, oval, etc.

In a further embodiment of the invention, the housing may be modified in its shape and dimensions. It is conceivable that, to accomplish this, the housing might be configured in the form of modules that could be connected to each other by plugging them together. Of course it is also conceivable that individual elements of the housing could be configured to be pulled out and pushed in, for example in a telescoping manner, and in this way change the dimensions and shape of the housing in order to generate a desire field. A desired number of plasma generators can be inserted in recesses in the housing, or they can already be permanently installed in the housing.

In a further embodiment of the invention, the apparatus may comprise a suction device in order to generate a stable, homogeneous field or even a laminar flow.

For example, suction nozzles that preferably are essentially located opposite the plasma generator nozzles may be provided in the housing to accomplish this.

According to the invention, for an operation, examination, or any desired treatment the apparatus is positioned close to the object, in particular to the person, or is placed in direct contact (contact with the object surface, in particular with the skin) so that the entirety of the sterile area that is generated in the surface covers the desired operating field. When this is done, the operating field generally only extends across a partial area of the surface of the body in which treatment is performed and/or an operation is performed through an opening into the interior of the body. On the basis of a field thickness of at least several millimeters or centimeters, a sterile field itself can be ensured in this way for a desired operation, i.e. this area may be sterilized.

If the apparatus or the plasma field generated by the apparatus is located—preferably in a plane parallel to the given area of the body surface—in direct contact with the skin, or if a possible intermediate space between the field and the skin is covered with a sterile covering that is impervious relative to the environment, for example a film, cover, etc., the sterility of a surgical opening can be maintained, even during an invasive intervention. In this way, the entry of contamination, in particular bacteria, into the unprotected interior of the body can be prevented in a simple manner.

As the plasma generator or plasma generators, it is possible to use generators that generate a plasma jet of a plasma or plasma gas—referred to below as a plasma gas—that (after being generated and emerging from a nozzle of the plasma generator) has a temperature of less than or equal to 40° C.—a so-called cold plasma gas. A plasma or a plasma jet that is too hot to treat a human being is cooled as needed by suitable cooling measures (cooling coils, cooling ribs, the addition of a cool gas, etc.) before it leaves a nozzle.

Based on the near or even the direct use and the corresponding arrangement of plasma generators in the area, it is possible to use standard plasma generators to generate a cold plasma, which preferably can at the same time be at atmospheric pressure (atmospheric pressure plasma) or at a higher pressure (high-pressure plasma).

The jet or flow of plasma gas—which preferably is generated from air, more preferably ambient air, and possibly with the addition of a carrier gas (such as argon) to achieve the effective range—can generate an antiseptic condition (preferably of the highest quality) in an area that is small (in comparison with the sterile field), and, based on the low required discharge rate and/or the laminar flow (with suction) the amount of ozone that occurs is relatively low and not harmful to health (low-ozone plasma gas). A sterile area or a sterile field that is generated in this way therefore is neither hazardous nor harmful to health for a human being, animal, or to sensitive surfaces of objects.

Moreover, by limiting the sterile field to an area that is essentially smaller than a body lateral surface of a person, a plasma gas which, were it released over a relatively wide area extent and therefore at a relatively high concentration in a closed room, could have an effect that would be harmful to human health (ozone concentration, pressure, temperature, etc.) can advantageously be used.

With regard to the type of plasma gas generators, therefore, all types of plasma generation could be used, in particular non-thermal plasma, with which, at least with use that was spatially limited in this way, a health hazard to a person or a patient (excessively high ozone concentration, excessive heat, excessive pressure, etc.) could be avoided.

Moreover, to reduce the risk of a health hazard, an undesirably high ozone concentration conceivably could be reduced or prevented in the plasma or the plasma jet through the use of suitable filters, such as HEPA filters (high-efficiency particulate air filters), ULPA filters (ultra-low penetration air), and SULPA filters (super ULPA), etc. These filters can also be used in the gas stream feed (air, in particular) to the plasma generators (before the plasma is generated) in order to prevent the induction of dust, particles, or microorganisms.

Generators that only generate a single plasma jet or area with a low depth may also be used advantageously through the utilization of a plurality of plasma generators because a field diameter can be covered with two generators that are located opposite each other and that have half of the plasma field depth (diameter/2).

In a preferred embodiment of the invention, the sterile area is generated by using a plurality (at least 2, 3, 4, 5, 6, or more), in particular plasma generators that are disposed in an open or closed housing that at least partially surrounds the field or the area in such a way that plasma or plasma gas is emitted into the interior of the field, preferably essentially within the same plane (coplanar). Of course, it is conceivable based on the limitation of the operating field in the downward direction to arrange individual or all plasma generators or their outlet nozzles at a slightly downward angle, or to configure them to be pivotable, so that any depressions in the operating field can be reached faster and more easily.

By using a plurality of plasma generators it is possible in a simple and economical manner to generate an area or a field with a sterilizing effect, in particular a medically (continuously) sterilizing effect. Standard plasma gas generators that generate a jet or a flow of cold plasma (gas)—preferably using air, more preferably ambient air—may also be advantageously used as plasma generators.

Such a field 0 can, for example, cover a surface that is several centimeters in diameter or whose sides are several centimeters apart, for example 10 to 60 cm, preferably 20 to 30 cm, and preferably at least 25 or at least 30 cm. An additional carrier gas (in particular argon) can be added to generate a field 0 having a relatively large diameter (depending on the shape of the field, a mean or maximum diameter), for example essentially larger than 50 cm or 60 cm—for example by means of a blower device, in order to increase the range of a plasma jet from a plasma generator.

In its depth or thickness, the field or the area can have a desired sterilizing effect and therefore a corresponding intensity within from (one to) a few millimeters up to a plurality of centimeters, for example 5 to 20 cm, preferably 1 to 10 mm (laminar flow).

In an more preferred embodiment of the invention, during a surgical intervention or a treatment, the work is preferably from the top or exterior through the sterile field that is generated in the direction of the body of a person who is to be treated, so that preferably also surgical instruments and hands or gloves are sterilized (again) when passing through the field.

In this medical application, the sterile field offers reliable protection against the entry of undesirable non-sterile particles, such as living organisms, spores, germs, viruses, in particular multi-resistant bacteria, in the immediate vicinity of a person, even during an invasive intervention and, hence, penetration of the protective skin layers. In this way, the risk of infection that exists in treatments and in particular in operations can be significantly reduced.

With the apparatus of the invention, the residual content of microorganisms that are capable of reproducing can be reduced in the sterile field to 10⁻⁷ or even 10⁻⁸ colony-forming units, therefore significantly below the required value of 10⁻⁶.

In a further embodiment of the invention, the apparatus has a movable arm on which the housing is pivotally arranged, preferably about an axis in the field plane, preferably with the aid of articulated joints. By means of this arm
or this support, the apparatus can be moved into any desired positions, vertically, and/or horizontally, and/or axially, so that the broadest possible range of operating fields can be covered.

In a further embodiment of the invention, the field may have a variable size and/or shape. To accomplish this, variously configured housings that preferably can be exchanged on the arm may be provided, for example, in which a corresponding number of plasma generators is used or is already permanently installed. Of course, it is also conceivable to configure the housing to be variable, even in its dimensions, for example movable, insertable, etc., whereby depending on the size that is set, a correspondingly varying number of mountings for plasma generators may be provided.

In a different application of the apparatus explained above, the apparatus may be used to sterilize persons, for example in an airlock.

To accomplish this, the apparatus is gradually moved across the entire body, for example in the form of a scanning operation, for example from the top down or vice versa, so that the field gradually sterilizes every (exterior) area of a person. In this case, the field may be transported perpendicular to the axis of the person's body by moving or traversing (scanning) the apparatus.

In a preferred embodiment of the invention, the housing is configured as a circumferential ring of any desired shape, in particular a circular ring, and possibly with a partial area that can be opened and closed.

Additional preferred embodiments of the invention may be derived from the dependent claims.

The invention is explained in greater detail below based on the embodiment example shown in the drawing. The drawing shows:

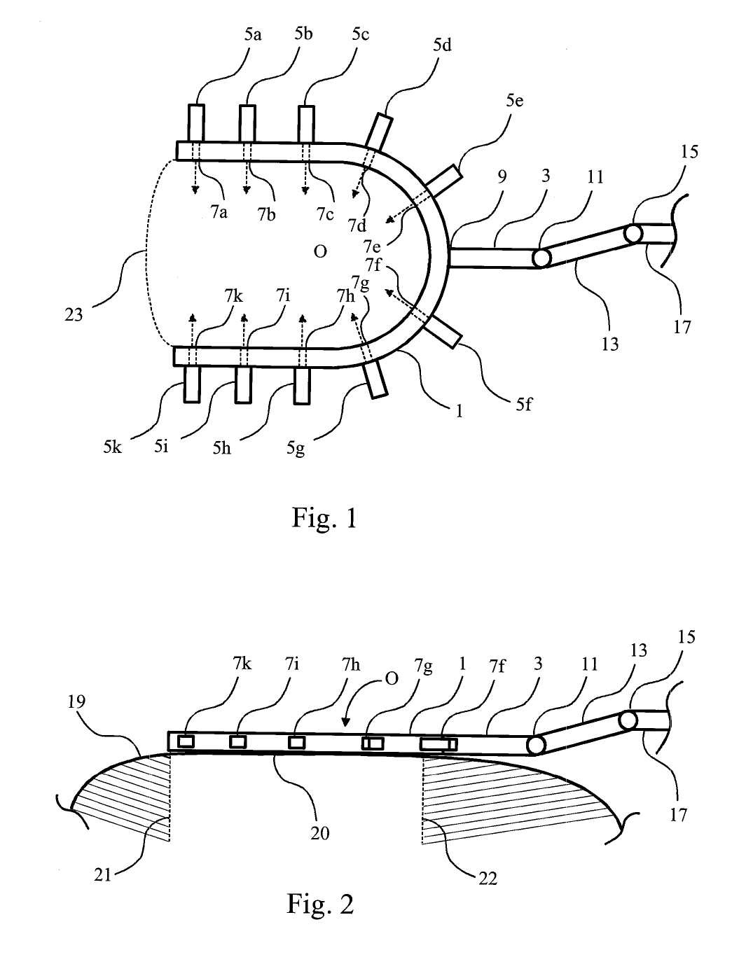

FIG. 1—a schematic side view of a closed space with an apparatus of the invention for sterilizing an operating area;

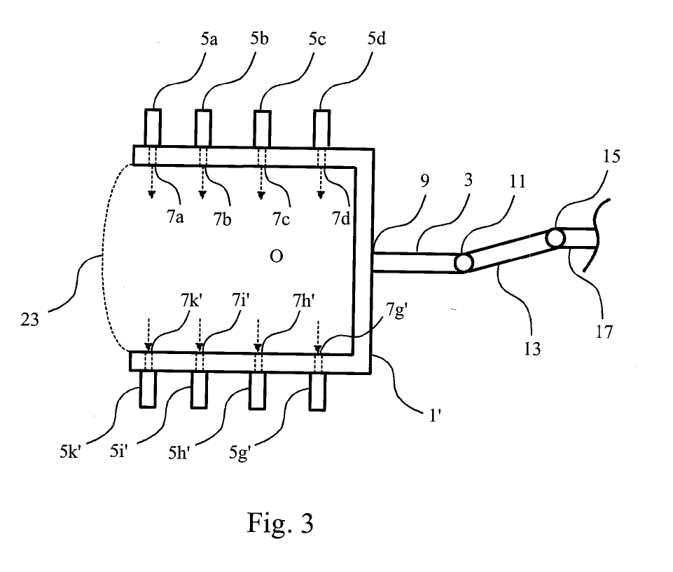

FIG. 2—a schematic view (outline) of a closed space in the form of an airlock having an apparatus of the invention to sterilize a person located therein; and FIG. 3—a schematic side view of a closed space with a modification of the apparatus of the invention set forth in FIG. 1.

The apparatus shown in FIG. 1 comprises a U-shaped housing or support 1 that is arranged to rotate by means of a preferably articulated connection 9 with a first partial area 3 of a support arm in the longitudinal axis of this partial area 3 and/or is arranged to pivot in the horizontal and/or vertical plane. A second partial area 13 is connected to this partial area 3 by means of a first articulated joint 11, and a third partial area 17 is connected to said second partial area 13 by means of a second articulated joint 15. Articulated joints 11 and 13 can be configured to rotate or pivot within the plane of the drawing and/or a plane that is perpendicular to the plane of the drawing. The support arm can also be made of fewer or more partial areas as needed, in order to achieve a required position.

In this way it is not only possible for a person to push the apparatus as a whole to a desired position in the room (on rollers or wheels, for example), but it is also possible to freely align the direction of the housing 1, and thus the sterile field 0 in all degrees of freedom.

The support arm may be part of a support that is securely anchored to the floor, or it may be moved, for example by means of rollers. The apparatus can be configured to be stationary or also standalone (case for setting up) with a standalone energy supply (rechargeable battery).

Of course it is also conceivable to anchor one end of the support arm securely to a wall.

In this way, the housing can be comfortably moved to a desired position, and the articulated joints are configured in such a way that the apparatus remains in a position or in addition can be secured in a position
that is set by hand for example—similar to a pivoting desk lamp.

Connections for the plasma generators 5a to 5k and a suction device, should one be present, as well as for an optional additional supply of air/gas or argon to the plasma generators can be provided to the plasma generators from the outside in a manner that is not shown in detail in the drawing. Of course, it is also conceivable, though, to provide appropriate supply feeds or lines within the housing 1 (hollow cavity construction).

FIG. 2 shows the apparatus or the housing 1 in a position above an abdominal wall 19 of a person or a patient who is lying perpendicular to the plane of the drawing (on an operating table, for example). As is shown in FIG. 2, the housing is in direct contact with the abdominal wall 19 (direct contact with the skin surface) or just above it. In its interior the housing 1 has a plasma field 0, which has a field edge 23 on the open side, which is shown as a dashed line.

A physician can treat the abdominal wall 19 from above through the plasma field 0, or can even open the abdominal wall 19, without causing germs to reach the abdominal wall 19, or without said germs entering the interior of the body. Hands, gloves, devices, etc. that enter the field 0 are also sterilized (after a sterilization that is customarily performed) so that not only the entry of germs from the environment (air) but also even via the operating surgeon are prevented. The plasma field 0 has a certain thickness in the middle (formation of convex shape) of several centimeters, for example, in particular from 2 to 5 cm, or up to 10 cm, preferably 0.5 to 2 cm, with a minimum thickness (on the field sides) of at least 1 mm or 1 cm.

In addition, a cover, which is not shown in greater detail, can be provided around the housing 1, covering or even sealing to the outside, in the form of a film or a cloth in order to cover the area outside of an area that is to be treated, in particular the operating area
20 or even to seal the operating area 20 or the exterior of the housing 1 relative to the abdominal wall 19. In the case of abdominal surgery, an operating area 20 and an opening in the abdominal cavity, which is schematically represented in FIG. 2 by dashed lines 21, 22, can be kept free of germs, and at the same time surgery can be performed through the plasma field 0 that covers the operating area 20.

As can be seen from FIG. 1 and FIG. 2, plasma generators 5a to 5k, which are disposed in recesses 7a to 7k of the housing 1 and which emit plasma gas into the field 0 in an inward direction in the direction of the arrow, are located on the housing.

In opposing legs of the U-shaped housing 1, plasma generators 5a, 5b, 5c are disposed opposite plasma generators 5k, 5i, and 5h so that the depth of the respective plasma gas jet is present in sufficient concentration to the center of the field, to generate a plasma field that has a sufficient sterilizing effect or sufficient concentration of plasma, in particular for an operation (abdominal surgery, for example).

The plasma generators 5a to 5k preferably admit plasma gas within the same plane of the field 0 (in FIG. 2 perpendicular to the plane of the drawing), whereby an inflow that is oriented in such a way can be achieved in a simple manner by aligning each nozzle of a plasma generator 5a to 5k accordingly. The nozzles can also extend through the recesses 7a to 7k or they may be formed

by the recesses themselves. In order to be able to include depressions in the operating field or in the field 0 better and faster, the plasma generators or their nozzles may also be inclined downward slightly or, if needed, they may be pivoted (by means of a pivoting apparatus). As a result of the descent of the plasma gas that is generated, depressions in the field 0 are quickly and reliably wetted with plasma gas, even with a coplanar inflow.

As is shown in FIG. 3, it is also conceivable to arrange at least one suction device 5k′, 5i′, 5h′, 5g′ with suction nozzles 7k′, 7i′, 7h′, 7g′ instead of opposing plasma generators 5a, 5b, 5c to 5k, 5i, and 5h, located opposite to plasma generators

5a, 5b, 5c (for example instead of plasma generators 5k, 5i, 5h, 5g) or vice versa. A rectangular housing 1′ that is open on the front side is shown as the housing shape in FIG. 3. Other basic shapes are, of course, also possible. The additional elements in FIG. 3 correspond to the elements already described in FIG. 1, so that identical reference numbers were used for them.

Individual plasma generators, as illustrated for example in FIG. 3 by reference numbers 5e-5f, may also be absent in various embodiments—depending on the geometry of the field 0—because plasma gas can already be generated sufficiently by plasma generators 5a-5d (and possibly 5k, 5i, 5h, 5g) on one or both preferably straight side(s). The suction can also be produced by a single suction device, which is not shown in the drawing and which supplies the necessary vacuum to all of the nozzles 7g′, 7h′, 7i′, 7k′. The connections (hoses, for example) that are used for this purpose may be routed to a shared suction device outside of the housing 1. Of course it is also conceivable to provide a hollow passage, for example a side configured as a hollow tube, in housing 1 as a connection between nozzles 7g′, 7h′, 7i′, 7k′ and the suction device. Of course it is also conceivable in all of the embodiments to route the plasma gas to the nozzles through a hollow passage, for example through a side that is configured as a hollow tube. In this case at least one plasma generator may be disposed at a location that differs from that shown in the drawing.

In all of the embodiments of the invention, a stable, homogeneous field or even a laminar flow can advantageously be generated. Here a plasma gas flow rate of several meters per second, preferably 2-10 m/s, more preferably 4-5 m/s, can be maintained from the outlet nozzle of a plasma generator 5a, 5b, 5c as a result of the laminar flow essentially across the entire field 0.

This advantageously results in a particularly homogeneous field 0 (the formation of turbulence is avoided) without interfering with an operating surgeon and without the operating area drying out in any noticeable manner. To generate a laminar flow, plasma generators 5a, 5b, 5c or their nozzles, preferably parallel to each other (more preferably coplanar or tangential to the surface to be treated), emit plasma gas into the field 0. Of course 4, 5, 6, 7, 8, or more generators can be arranged for this purpose on one side and preferably can be switched on as needed.

In a complementary manner, opposing (parallel) suction nozzles 7g′, 7h′, 7i′, 7k′
that remove the plasma gas in a manner that is not shown in greater detail (discharge at a distance from field 0, deionize, filter, in particular by means of an active carbon filter or recombination filter, temporarily store, discharge the gas or air in a non-ionized state) can be arranged on the other side (in particular coplanar or tangential to the surface being treated). In particular in a field 0 having laminar plasma gas flow, depressions in the field 0 are reached, respectively wetted by the (descending) plasma gas. Likewise, prominences are also reached by the flow. Here the laminar flow follows the hills and valleys in the field 0.

In any embodiment of the invention, the plasma field 0 can have a field size with a mean diameter (in particular the distance between the sides) of from 10 cm to 60 cm, preferably up to 30 cm or 40 cm, depending on what is required.

To achieve different sizes and/or shapes, it is also conceivable to configure the apparatus, in particular the housing 1 itself, so that it can be changed, for example so that it can be pulled out, pushed together, etc. For example it is conceivable to be able to pull out and push in in a telescoping manner the straight side areas of the housing 1 that are shown in FIG. 1, whereby in the extended condition—compared with the pushed-in (shortened) condition—additional recesses may be provided in which, as needed, additional plasma generators may be used to generate plasma having a sufficient concentration for a sterilization in the enlarged field 0.

In any given embodiment of the invention, depending on the number of microorganisms that are present, a desired sterility (remaining content of microorganisms capable of reproduction) in the sterile field of 10⁻⁶, preferably less than 10⁻⁷, or even less than 10⁻⁸ colony-forming units can be achieved after a few seconds or after a number of minutes, for example from 20 sec to 15 min.

Here only a low amount of ozone is formed, with a concentration of less than 0.1 ppm, preferably less than 0.5 ppm, or less than 0.3, in particular of 0.028 ppm, even in the immediate vicinity of the field 0. In field 0, in the various embodiments of the invention, a concentration of positively charged electrons from 10⁹ to 10¹² is present.

In a particular embodiment of the invention, only ambient air is used to generate the plasma gas, so that it is not necessary to supply additional (carrier) gas, in particular argon.

The field 0 with its specified size (for example an area of at least 400, 600, 700, 1000, or 1500 cm²) is completely filled by plasma or plasma gas with an (average) thickness of several centimeters, in particular at least several millimeters, 1 cm, 2 cm, 3 cm, or more. In a field 0 with preferably laminar flow, it is also possible to generate a continuous field having a low thickness of at least 1 mm on the sides and extending to a center (formation of a convex shape with laminar flow in the middle or tapering in the direction of the nozzles) having at most 10-25 millimeters with an area of 30 cm×30 cm, without the flow failing and non-sterile areas resulting in the field.

As described above, a sterilizing plasma field 0 can be generated as needed by the plasma generators 5a to 5k. The emitting of plasma (gas) into the field 0 or into the sterile area can be accomplished by the plasma generators in a pulse-like manner or continuously. An adequate concentration of plasma in the field can also be monitored, for example, by means of a sensor, and, if necessary, it can be logged for later use. Such a sensor can be provided directly on the housing, for example, or it can pivot by means of an arm into the field 0.

With a known distribution of plasma in field 0, the sensor may be disposed at any given position within the field 0 so that, if the distribution is known, a minimum threshold value at the location of the sensor can be preset in order to detect when a sufficient concentration of plasma is reached or even to ensure that the concentration is monitored.

The apparatus of the invention is advantageously also used to achieve a re-sterilization of gloves and instruments that come in contact with,
enter, or pass through the field 0. In this way the entry of germs, bacteria, fungi, spores in the treatment area, in particular the operating opening, can be prevented. Moreover, instruments (all together, even on the contacting side) can be placed in an area of the field 0 that is not required for the operation and can be kept sterile there. For this purpose it is possible to provide webs, bowls, nets, etc. that, for example, can preferably be articulated on the support, pivoted into or arranged in the field 0 (set onto, inserted, etc.) as needed.

Of course the invention is not limited to the embodiment example that is shown. For example, the invention may also be used to implement plasma sterilization in an (access) airlock for persons. To accomplish this, it is possible to preferably move a circumferential annular housing having a plasma field 0 perpendicular to the body axis from above and/or below along a person or an object in order to sterilize the person or object. An airlock of this type may be used, for example, ahead of an entrance to rooms (operating room) or buildings (clinics, hospital departments) in which an environment that is already for the most part sterile is present or in which further contamination with external germs is to be prevented. It is also conceivable to use such airlocks in front of public buildings that have high visitor rates and therefore a high risk of infection (train stations, government buildings, businesses, etc.) or also for private homes and residential buildings to prevent external contamination and the resulting risk of infection.

### LIST OF REFERENCE NUMBERS

0 Plasma field
1, 1′ U-shaped housing/support
3 Support arm, first area
5a-5k Plasma generators
7a-7k Recesses/through-holes
5g′-5k′ Suction devices
7g′-7k′ Suction nozzles
9 Connection between support 1 and arm 3
11 First articulated joint
13 Support arm, second area
15 Second articulated joint
17 Support arm, third area
19 Abdominal wall
20 Partial area of the abdominal wall 19 to be treated/operated on
21 Edge (does not need to be a straight) of the sterile area/operating area (protected from the entry of germs/bacteria into the zone below the surface)
22 Edge (does not need to be straight) of the sterile area/operating area (protected from the entry of germs/bacteria into the zone below the surface)
23 Front boundary of the plasma field 0

1. A method for generating a sterile area for an operation, examination, or treatment of at least one partial area of an object, wherein cold plasma gas is emitted into the area using at least one plasma generator.

2. The method of claim 1, wherein an essentially planar sterile field, which is essentially parallel or vertical to the partial area of an object, is generated as the area.

3. The method of claim 1, wherein the sterile area is generated by means of a plurality of plasma generators, and the plasma gas is emitted in an essentially coplanar manner into a sterile field.

4. An apparatus for carrying out the method of claim 1, wherein the apparatus has at least one plasma generator by which cold plasma gas is emitted into the area as needed.

5. The apparatus of claim 4, wherein the apparatus has, at the edge of the area, at least a partially surrounding housing in which a plurality of plasma generators are arranged in such a way that they emit plasma gas in a direction of an interior of the area as needed.

6. The apparatus of claim 5, wherein the plurality of plasma generators are arranged in such a way that they generate a planar sterile field.

7. The apparatus of claim 5, wherein the housing is U-shaped.

8. The apparatus of claim 5, wherein the housing is closed in the shape of a ring.

9. The apparatus of claim 5, wherein the apparatus has a housing whose shape and dimensions can change.

10. The apparatus of claim 4, wherein the apparatus comprises a suction device configured to generate a stable homogeneous field.
